(19) United States
(12) Patent Application Publication
(10) Pub. No.: US 2023/0404436 A1
(43) Pub. Date: Dec. 21, 2023

(54) HYBRID WALKING ANALYSIS APPARATUS FOR FALL PREVENTION AND FALL PREVENTION MANAGEMENT SYSTEM COMPRISING SAME

(71) Applicant: OPTONICS CO., LTD, Gwangju (KR)

(72) Inventors: Youngwoo LEE, Gwangju (KR); Yookang JI, Gwangju (KR); Jaehong JOO, Gwangju (KR)

(21) Appl. No.: 17/911,236

(22) PCT Filed: Nov. 20, 2020
(86) PCT No.: PCT/KR2020/016474

§ 371 Date: Sep. 13, 2022

(30) Foreign Application Priority Data
Mar. 13, 2020 (KR) 10-2020-0031268

(51) Int. Cl. A61B 5/11; G06T 7/00

(52) U.S. Cl. CPC A61B 5/1117; G06T 7/00; G06T 2207/10028; G06T 2207/30008

(57) ABSTRACT

Provided is a hybrid walking analysis apparatus for fall prevention and a fall prevention management system including same and, wherein the apparatus provides a measurement system based on an examinee's walking characteristic, and performs a customized analysis using collected information so as to enable fall prediction and prevention.

### TECHNICAL FIELD

The present invention relates to a hybrid walking analysis apparatus for providing a measurement system based on walking characteristics of a subject and performing a customized analysis using collected information to enable fall prediction and prevention, and a fall prevention management system including the same.

### BACKGROUND ART

In general, falls are caused by complex factors, such as physical, psychological, environmental factors, and the like, and thus an integrated approach including socio-psychological factors is required.

There are various factors that cause falls, and because a higher number of factors leads to a higher risk of falling, there is a need for a screening test to assess the risk of falling.

Currently, the most widely used fall risk assessment includes ‘receiving a fall history’ and ‘assessment of gait and balance.’

However, most of the assessment processes exclude physical factors and measure psychological factors, such as knowledge, self-efficacy, preventive behavior, and depression, and thus have a limitation in testing the power of exercise effect.

When body balance and walking ability are measured without using measuring equipment, errors can occur due to the subject's will or motion during a measurement process, and subjective opinions can be excessively included depending on the examiner.

In some measurements, although equipment for measuring physical abilities is used, since a one-sided measurement method with limited content has limitations and targets the general public, not the elderly, there is a need to develop a system that meets the characteristics of the elderly and user experience (UX).

### RELATED ART DOCUMENTS

### Patent Document

(Patent document 1) Korean Patent Publication No. 10-2019-0070068

(Patent document 2) Korean Registered Patent No. 10-1713263

### DISCLOSURE

### Technical Problem

The present invention has been proposed to solve the above-described problems, and provides a hybrid walking analysis apparatus for providing a measurement system based on walking characteristics of a subject and performing a customized analysis using collected information to enable fall prediction and prevention, and a fall prevention management system including the same.

### Technical Problem

To this end, a hybrid walking analysis apparatus for fall prevention according to the present invention includes: a walking analyzer (11) installed on a floor on which a subject of a fall prevention test walks, and provided with a plurality of pressure sensors for measuring a foot pressure during walking to provide foot pressure information; a depth camera (12) installed to photograph the walking subject and providing a three-dimensional (3D) depth image of the subject; and a walking analysis module (13) configured to synthesize the foot pressure information of the subject and skeletal information read from the 3D depth image to analyze a walking state of the subject.

In this case, the hybrid walking analysis apparatus may preferably further include an automatic user recognizer (14) configured to investigate a position of the subject to analyze each of a point in time when the subject enters the mat-type walking analyzer (11) and a point in time when the subject leaves the mat-type walking analyzer (11).

In addition, the walking analyzer (11) may preferably include a plurality of unit mat sensors (C1 to Cn), each of which is provided in a mat type, wherein each of the unit mat sensors (C1 to Cn) may include a plurality of pressure sensors arranged in a matrix pattern.

In addition, each of the unit mat sensors (C1 to Cn) may preferably include: a floor sheet (11a); a first sensor sheet (11b) stacked on an upper surface of the floor sheet (11a) and provided with a horizontal axis pressure sensor in a horizontal direction; a conductive material layer (11c) stacked on an upper surface of the first sensor sheet (11b) and formed of a conductive material; a second sensor sheet (11d) stacked on an upper surface of the conductive material layer (11c) and provided with a vertical layer pressure sensor in a vertical direction to intersect the horizontal axis pressure sensor; and a finishing sheet (11e) stacked on an upper surface of the second sensor sheet (11d).

In addition, the depth camera may preferably include at least one depth camera (12) installed along the walking analyzer (11) and may preferably be configured to continuously photograph the subject within designated sections.

In addition, the hybrid walking analysis apparatus may preferably further include a two-dimensional (2D) inspection camera (15) configured to photograph the subject as a 2D red-green-blue (RGB) image and provide the 2D RGB image to the walking analysis module (13), wherein the walking analysis module (13) may be configured to extract, among image frames in which the subject is photographed, an error frame that is different from an actual walking state using an analysis result of the 3D depth image provided by the depth camera (12) and an analysis result of the 2D RGB image provided by the inspection camera (15) and perform interpolation.

In addition, the walking analysis module (13) may preferably be configured to extract images of a skeleton and joints of the subject from an image frame through machine learning, wherein the walking state of the subject including a position value and an orientation value of the skeleton and the joints may be analyzed.

In addition, the walking analysis module may preferably include: an object extraction unit (13a) configured to extract a walking aid used by the subject during walking in the 3D depth image; a noise removing unit (13b) configured to exclude the extracted walking aid from the image frame to be analyzed; a walking analysis unit (13c) configured to analyze the walking state of the subject using the image frame from which the walking aid is excluded; and a fall prediction unit (13d) configured to compare the walking state of the subject with a fall risk factor to predict a risk of falling.

Meanwhile, a fall prevention management system according to the present invention includes: the hybrid walking analysis apparatus (10) of any one of claims 1 to 11; an integrated database (20) configured to record personal information and fall test history information of a subject; and an app management module (30) configured to provide an application that allows a terminal device of the subject to access the integrated database (20) and receive a test result.

In this case, the integrated database (20) may preferably be configured to provide the subject with a customized fall prevention program suitable for the subject.

### Advantageous Effects

As described above, the present invention can provide a hybrid type measuring apparatus that comprehensively reflects foot pressure measurement and skeletal information during walking while performing the measurement in a state in which a subject who is to be tested is not aware of the measurement of the walking.

In addition, the present invention can provide subject's personal information and fall test history information in real time through an integrated database, and also provide a fall prevention program optimized for an individual test group.

Therefore, a measurement system based on walking characteristics of a subject is provided, and a customized analysis using collected information is performed, and thus an integrated system capable of predicting and preventing falls can be provided.

### DESCRIPTION OF DRAWINGS

FIG. 1 is a schematic block diagram of a fall prevention management system according to the present invention.

FIG. 2 is a block diagram illustrating a hybrid walking analysis apparatus for fall prevention according to the present invention.

FIG. 3 is a diagram illustrating a mat-type walking analyzer array according to the present invention.

FIG. 4 is a stacked state diagram of a mat-type walking analyzer according to the present invention.

FIG. 5 is a diagram illustrating a method of measuring the position of a subject using a received signal strength indicator (RSSI) according to the present invention.

FIG. 6 is a diagram illustrating a method of measuring the position of a subject in triangulation using a beacon according to the present invention.

FIG. 7 is a diagram illustrating skeletal information during walking acquired by a depth camera according to the present invention.

FIG. 8 is a diagram illustrating a state in which an error frame is removed in the present invention.

FIG. 9 is a block diagram illustrating a walking analysis module according to the present invention.

FIGS. 10A and 10B are diagrams illustrating a method of extracting walking information from a captured image in the present invention.

FIG. 11 is a diagram illustrating a method of recognizing a unit detection sensor during walking measurement according to an embodiment of the present invention.

### MODES OF THE INVENTION

Hereinafter, a hybrid walking analysis apparatus for fall prevention and a fall prevention management system including the same according to an exemplary embodiment of the present invention will be described in detail with reference to the accompanying drawings.

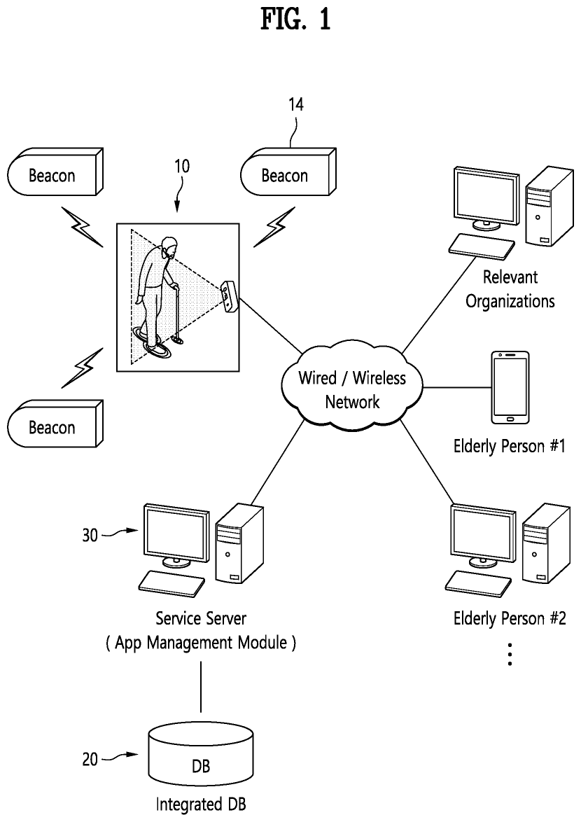

First, prior to detailed description of a hybrid walking analysis apparatus 10 according to the present invention, an embodiment to which fall prevention management systems (10, 20, and 30) including the hybrid walking analysis apparatus (10) is applied will be briefly described with reference to FIG. 1.

As shown in FIG. 1, the fall prevention management systems (10, 20, and 30) according to the present invention includes a hybrid walking analysis apparatus (10), an integrated database (20), and an app management module (30), which are connected to each other through a wired/wireless network.

The hybrid walking analysis apparatus (10) is a detection apparatus for predicting and preventing a fall of a subject, and measures the foot pressure during walking using a mat-type walking analyzer (11) and measures the skeletal movement during walking using a depth camera (12).

With a hybrid measurement using both the walking analyzer (11) and the depth camera (12), the subject's fall factors may be analyzed based on objective measurement data, and combination of the information may provide more various gait parameters.

In this case, the above-described subject is a subject who is in great need of fall prediction and prevention compared to the general public, such as an elderly person (senior citizen), a disabled person, or a person with activity problems, and the following description will be made in relation to an example of an elderly person.

In the integrated database 20, personal information and fall test history information of the elderly person who is the subject are recorded. The integrated database 20 receives measured data values from the hybrid walking analysis apparatus 10, and the measured data values are recorded therein and provided to the elderly person (the subject). In addition, the measured data values may be provided to related organizations as needed.

Such an integrated database 20 may be built alone, or may preferably be built together with a web server as a database server type. In addition, a service server may be built as a wireless application protocol (WAP) server or a cloud server to provide a service on a mobile terminal such as a smart phone.

The app management module 30 provides an application for a terminal apparatus of the elderly person who is the subject to access the integrated database and receive a test result, and the app management module 30 may be installed together in the above-described service server, for example. In this case, the service server functions as an application download server.

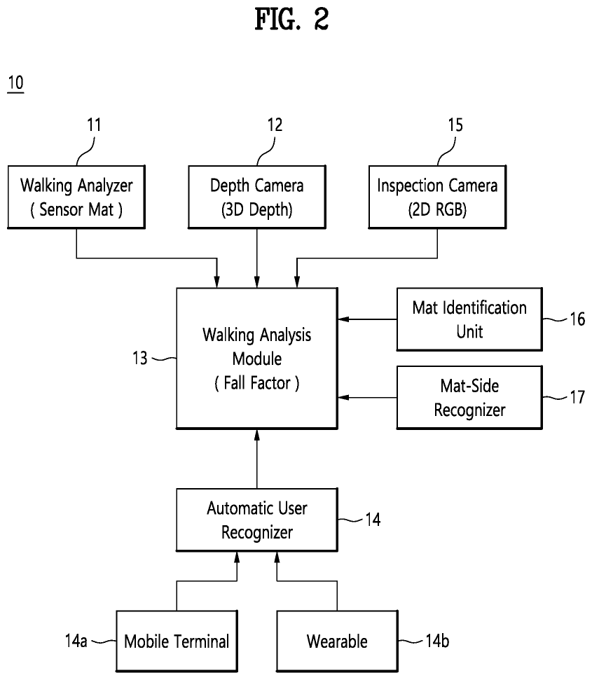

Hereinafter, the hybrid walking analysis apparatus 10 constituting the fall prevention management system of the present invention will be described in detail with reference to FIG. 2.

As shown in FIG. 2, the hybrid walking analysis apparatus 10 of the present invention includes a walking analyzer 11, a three-dimensional (3D) depth camera 12, and a walking analysis module 13. Furthermore, as another exemplary embodiment, the hybrid walking analysis apparatus 10 further includes an automatic user recognizer 14 and a two-dimensional (2D) inspection camera 15.

Here, the walking analyzer 11 measures the foot pressure of the elderly person corresponding to the subject, the depth camera 12 analyzes the skeleton of the walking subject through a 3D image, and the walking analysis module 13 synthesizes pieces of measurement information to analyze fall factors.

In addition, the automatic user recognizer 14 detects (investigates) the position of the elderly person who is the subject using short-range wireless communication and provides the position to detect whether the subject enters and/or leaves the walking analyzer 11 provided in a mat type, thereby increasing the test precision.

The 2D inspection camera 15 provides a captured image for analyzing the walking of the subject together with the 3D depth camera 12 described above. Therefore, through the advantages of 3D images and the advantages of 2D images, error frames different from an actual walking posture are found and excluded.

More specifically, the walking analyzer 11 measures the foot pressure of the subject of the fall prevention test, that is, the elderly person, during walking and also enables analysis of walking together with foot pressure.

To this end, the walking analyzer 11 is installed on the floor (e.g., a hallway or a measurement place) on which the fall prevention subject walks, and is provided with a plurality of pressure sensors at regular intervals to measure the foot pressure during walking.

In addition, the walking analyzer 11 includes a plurality of ‘unit mat sensors’ to provide ease of storage, transport, and assembly according to the environment. In this case, each of the unit mat sensors is manufactured in a mat type to guide natural walking of the elderly person.

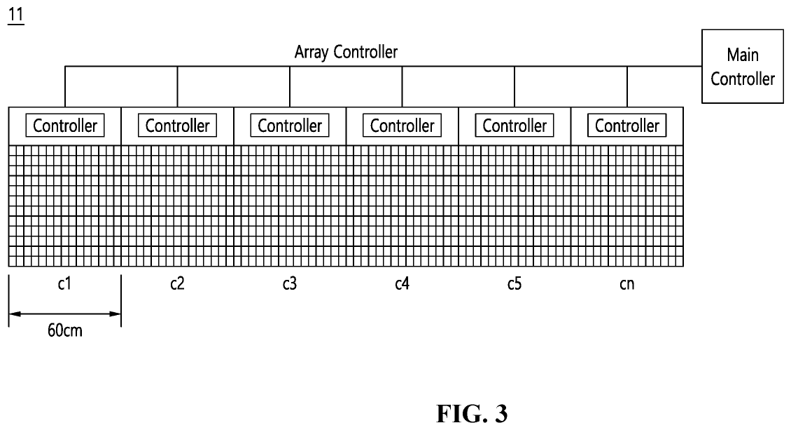

As shown in FIG. 3, the plurality of unit mat sensors C1 to Cn are manufactured to have, for example, a quadrangular cross-sectional shape, and by being connected in a line form to a main controller, provide measurement values measured at each pressure sensor to the main controller.

In addition, since the unit mat sensors C1 to Cn include a plurality of pressure sensors that are provided in a matrix pattern to have a distribution density greater than or equal to a certain level, a foot pressure measurement during walking allows intuitive measurement information such as center of pressure (CoP) and center of mass (CoM) to be provided, based on which walking analysis is performed.

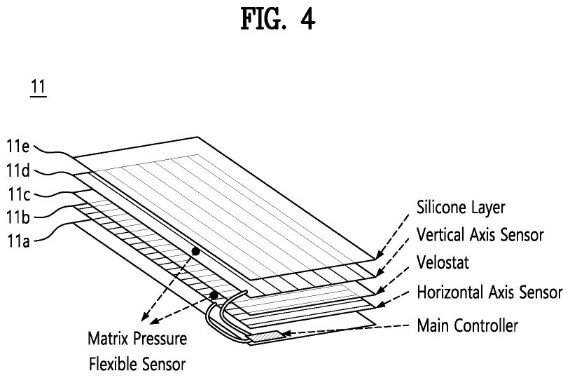

As shown in FIG. 4, the unit mat sensors C1 to Cn in a mat type, as an exemplary embodiment, have a multilayer structure including a floor sheet 11a, a first sensor sheet 11b, a conductive material layer 11c, a second sensor sheet 11d, and a finishing sheet 11e.

Of the multilayer structure, the floor sheet 11a is disposed on the lowest layer of the unit mat sensors C1 to Cn, and an anti-slip member or the like is provided on the bottom surface thereof. In addition, an array controller is provided on one side of the floor sheet 11a. The array controllers each provided in one of the unit mat sensors C1 to Cn are centrally connected to the above-described main controller.

The first sensor sheet 11b is stacked on an upper surface of the floor sheet 11a, and is provided with ‘horizontal axis pressure sensors’ in the horizontal direction. For example, the horizontal axis pressure sensors are manufactured by forming a linear pattern on a sheet.

Here, the ‘horizontal’ direction is a direction that intersects the ‘vertical’ of the second sensor sheet 11d to be described below, and the horizontal and vertical directions may differ depending on the position in which the sheet is placed in a plan view or in the viewing direction.

The conductive material layer 11c is stacked on an upper surface of the first sensor sheet 11b and formed of a conductive material. As the conductive material layer 11c according to an embodiment, a pressure sensing cloth referred to as ‘velostat’ may be used to measure a pressure value.

The second sensor sheet 11d is stacked on an upper surface of the conductive material layer 11c so that the first sensor sheet 11b and the second sensor sheet 11d are disposed with the conductive material layer 11c interposed therebetween, and is provided with ‘vertical layer pressure sensors’ in the vertical direction to intersect the horizontal axis pressure sensors of the first sensor sheet 11b.

Accordingly, the horizontal axis pressure sensors of the first sensor sheet 11b and the vertical axis pressure sensors of the second sensor sheet 11d intersect to form a matrix pattern, and simultaneously measure the foot pressure of the subject at each point of the sensors, and thus the above-described CoP and CoM are precisely measured.

In the above-described mat-type walking analyzer 11, as an embodiment, the matrix is set to have a minimum interval of 0.5″ (1.27 cm) so that areas of the foot pressure during walking are precisely divided and detected. In addition, in consideration of the step width, the size of each of the unit mat sensors C1 to Cn is designed to be 61 cm×61 cm (Width, Height).

Accordingly, each of the unit mat sensors C1 to Cn is configured to include 2,304 (48×48) pressure sensors. The total length of the mat may be provided in various sizes according to an array in which the plurality of unit mat sensors C1 to Cn are connected to each other.

The finishing sheet 11e is stacked on an upper surface of the second sensor sheet 11d, and for example, a silicone layer formed of a silicone material is used. Accordingly, the finishing sheet 11e prevents the elderly person who is the subject from slipping during measurement while absorbing a shock.

On the other hand, the automatic user recognizer 14 recognizes the position of the subject to analyze the times when the subject enters and leaves the above-described mat-type walking analyzer 11, for example, using short-range wireless communication (including near field communication) to recognize (investigate) the user.

The short-range wireless communication for recognizing a user (a subject) typically includes a Bluetooth Low Energy (BLE) protocol communication function provided by mobile terminal apparatuses 14a and 14b, and in this case, the automatic user recognizer 14 is a Bluetooth beacon.

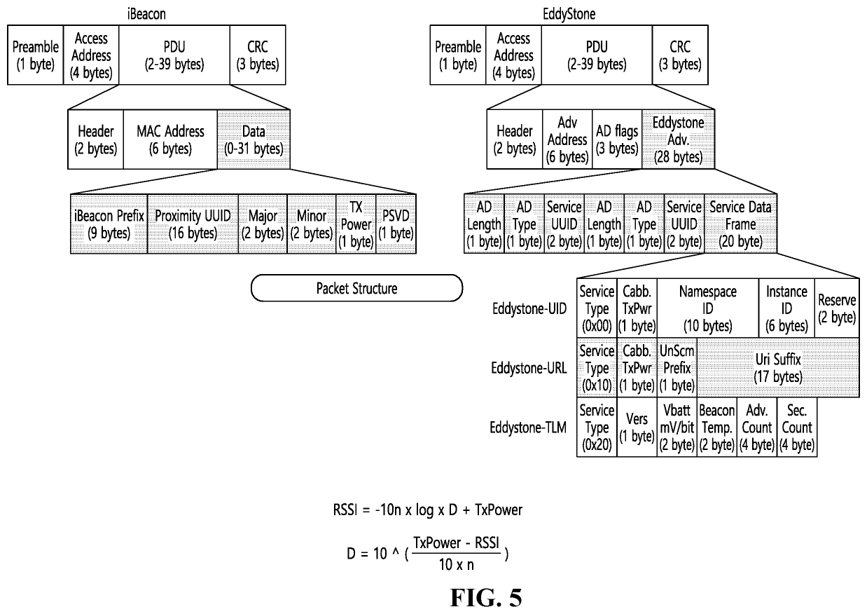

As shown in FIG. 5, the Bluetooth beacon receives a Bluetooth wireless signal transmitted from the smart phone 14a possessed by the subject or the wearable device (e.g., a smart watch or band) 14b worn by the subject, and the received data packet includes information about TxPower.

Therefore, a received signal strength indicator (RSSI) is calculated using the information included in the received data packet, and an approach of the subject may be recognized from the calculated RSSI value.

Therefore, since the test is started after recognizing the subject through the automatic user recognizer 14 in a state in which the subject is not aware of the start of the test, the subject may have the walking ability evaluated in a natural walking state as usual without recognizing that walking is being measured.

A device for recognizing a subject has been illustrated above as a short-range wireless communication device, and among them, a beacon for BLE communication has been described. However, as a short-range wireless communication device for recognizing a subject, other short-range recognition devices, including radio frequency identification (RFID) tags, may be used.

In addition, although the present invention takes an example of using an RSSI in a beacon as a method of recognizing a user, a trilateration algorithm may be used to compensate for the disadvantage of a beacon that it is difficult to obtain a constant RSSI value due to the propagation characteristics of a beacon.

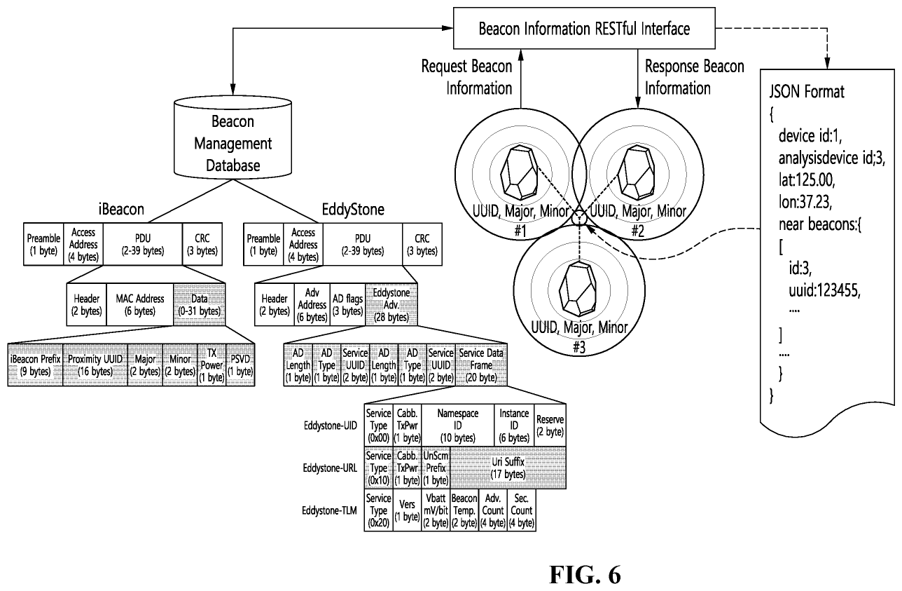

As shown in FIG. 6, the triangulation algorithm is implemented such that Bluetooth signals emitted from a signal source, such as a smart phone, are received by three different beacons and the reception time and reception direction are applied to the triangulation technique to predict the position of the user.

In this case, a Kalman Filter algorithm is used for performing normalization on RSSIs of the beacon, and the beacons are installed at the start and end points of walking measurement so that the recognition is achieved only with approximations near the start and end points and thus it is preferable to determine inclusion within a recognition threshold range, rather than determining an accurate distance value.

Next, the depth camera 12 installed to photograph a walking subject corresponds to a computational camera for providing a 3D depth image of the subject and calculates the depth value of each pixel, which has not been calculated with the existing 2D camera.

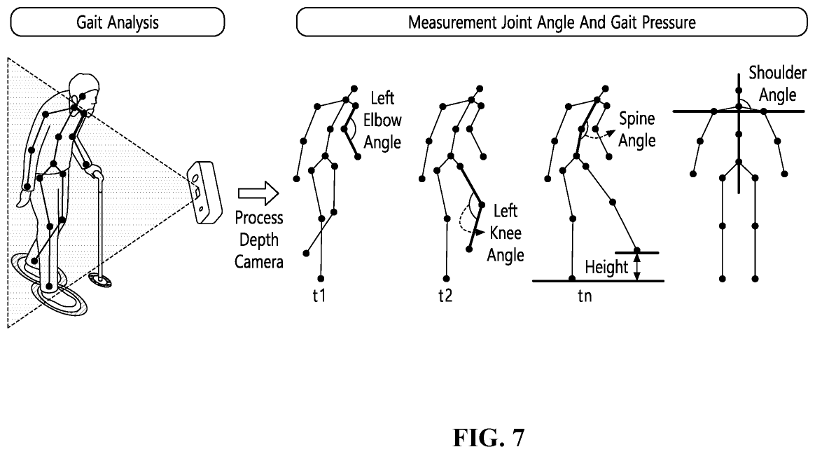

As shown in FIG. 7, the depth camera 12 may perform an operation on RGB, saturation, and contrast information of pixels provided by a 2D image as well as an image coming through a lens, to reprocess the image so that skeletal information of the subject is represented.

Therefore, when at least one depth camera 12 is installed along the walking analyzer 11 and continuously photographs the subject within the designated sections, skeletal information of the subject including the skeleton and joints may be provided.

For example, information about the position and the rotation angle, which is an orientation, of skeleton joints may be used to extract walking analysis parameters. In addition, through the skeletal information, various walking patterns and walking postures, including left-right balance, a leg angle, and a foot height during walking, may be analyzed.

However, the present invention preferably further includes the 2D inspection camera 15, wherein the inspection camera 15 photographs the subject as a 2D RGB image and provides the 2D RGB image to the walking analysis module 13. That is, the present invention provides a 2D photographed image together with the 3D depth camera 12 to analyze two images of different dimensions, thereby providing more accurate walking information.

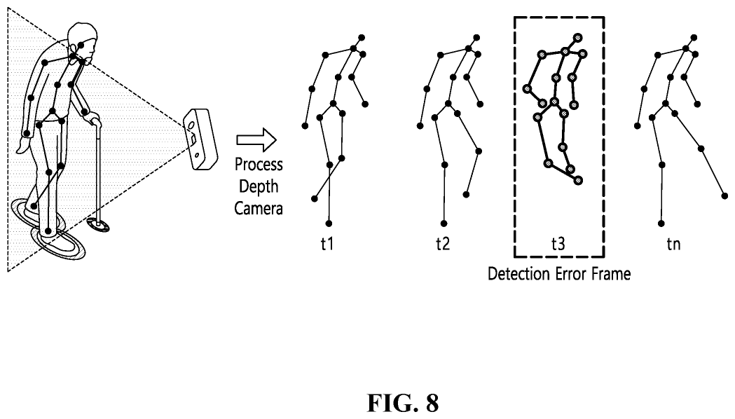

As an example, as shown in FIG. 8, the walking analysis module 13 uses an analysis result for a 3D depth image provided from the depth camera 12 and an analysis result for a 2D RGB image provided from the inspection camera 15 to extract error frames different from the actual walking state from among image frames of the subject. Furthermore, the walking analysis module 13 excludes or corrects the extracted error frame so that the image frames are interpolated.

That is, the skeleton is recognized based on 3D position and orientation values of each joint measured through the depth camera 12, but in this case, there is a high probability that an error may occur in recognizing the skeleton using depth values due to overlapping of the legs, arms, and body or surrounding objects depending on a field of view (FoV) and the angle of the camera.

Therefore, in order to reduce such errors, an algorithm and an interpolation algorithm for simultaneously analyzing a 2D RGB image and a 3D depth image to recognize the skeleton and also detecting an error frame of the depth image are applied so that the skeleton is recognized.

Next, the walking analysis module 13 synthesizes the foot pressure information of the subject and skeletal information read from the 3D depth image to analyze the walking state of the subject, thereby predicting and preventing falls of the elderly person.

In other words, through the skeleton information obtained by image analysis during walking of the subject, various walking patterns and walking postures, including left-right balance, a leg angle, and a foot height, and the like are analyzed to predict and prevent falls.

The walking analysis module 13 may be installed alone in a separate computing terminal apparatus, or may be integrally installed with the walking analyzer 11. In addition, the walking analysis module 13 may be installed in the service server and receive measurement data transmitted through a wired/wireless communication network and process a process.

In particular, the walking analysis module 13 extracts images of the skeleton and joints of the subject from the image frame through machine learning (ML). Furthermore, the walking analysis module 13 analyzes the walking state of the subject including the position and orientation values of the skeleton and joints.

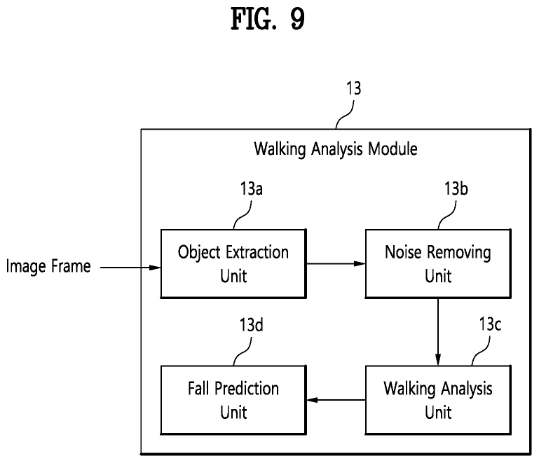

To this end, as shown in FIG. 9, the walking analysis module 13 includes an object extraction unit 13a, a noise removing unit 13b, a walking analysis unit 13c, and a fall prediction unit 13d to extract gait parameters.

Gait parameters are read from the user's walking step, stride, stride length, step width, single support, double support, flight time, step velocity, CoP, CoM, and the like by an algorithm.

Here, the object extraction unit 13a extracts a walking aid used by the subject during walking from the 3D depth image. The walking aid is a crutch, a cane, a walker, or the like used by the elderly person.

Therefore, since the walking aid needs to be excluded from analysis of the walking information of the subject, that is, the elderly person, an image representing the walking aid in the image frame is treated as a type of an object and removed as follows.

The noise removing unit 13b excludes (deletes or ignores) the walking aid extracted in the form of an object from an image frame to be analyzed, and thus only information related to the subject's walking is provided for the analysis.

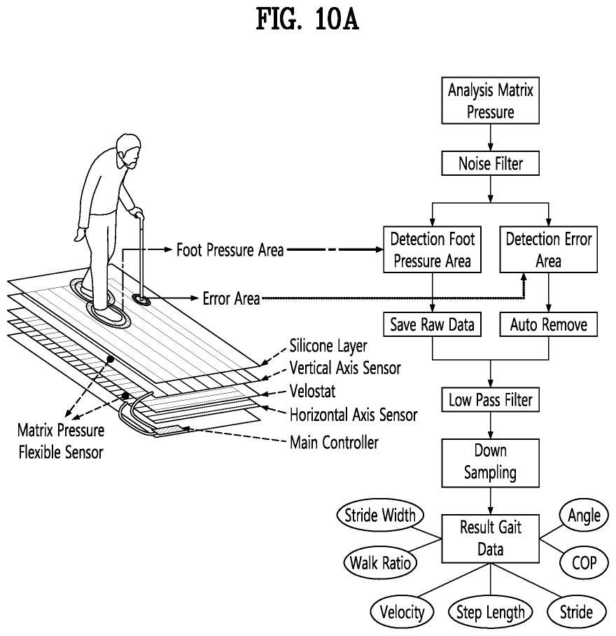

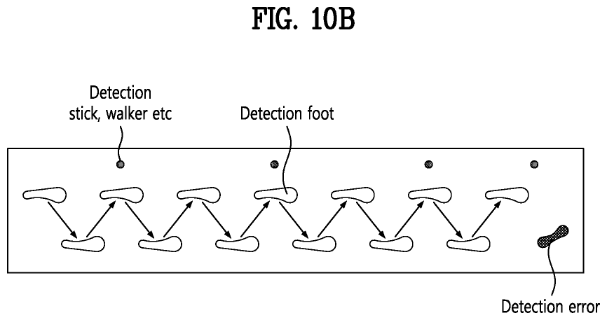

As shown in FIGS. 10A and 10B, when the subject walks on the mat-type walking analyzer 11, the pressure of an area generated by the foot of the subject and the pressure of an area generated by the walking aid are detected.

The pressure sensing value of the area generated by the subject's foot is stored as raw data and then down-sampled and analyzed so that various effective data are generated. On the other hand, the pressure value generated by the walking aid is removed by the noise removing unit 13b.

The walking analysis unit 13c uses an effective analysis target image frame, in which the walking aid is excluded as described above, to analyze the walking state of the subject, and the fall prediction unit 13d compares the walking state of the subject with a fall risk factor to predict the risk of falling.

That is, for the walking analysis, 3D position and orientation information of joints from skeletal information obtained through the depth camera 12 is received, the received information is combined to analyze the walking information of the subject, and thereby the risk of falling is predicted.

In addition, user walking analysis information is collected and analyzed through a fall test for the general elderly and fall risk groups, and an algorithm of extracting and processing fall risk factors and classifying risk groups is applied.

In addition, a classification/learning/prediction algorithm is applied through a walking pattern comparison model dataset, a fall risk group classification dataset, and a user-specific fall prediction dataset, and based on a fall risk group classification model and user personal walking pattern data, the risk of falling is predicted.

Hereinafter, another embodiment applicable to the hybrid walking analysis apparatus for fall prevention according to the present invention will be described.

The embodiment allows identification or arrangement analysis of each of the unit mat sensors C1 to Cn constituting the walking analyzer 11 to be reflected in the measurement of the subject's walking.

To this end, as shown in FIG. 2, the hybrid walking analysis apparatus for fall prevention according to the present invention further includes a mat identification unit 16 and a mat-side recognizer 17 as a technical configuration added for the embodiment.

The mat identification unit 16 allows own unique identification information to be provided (transmitted) from each of the above-described unit mat sensors C1 to Cn, wherein, for signal transmission, the unique identification information is loaded in a packet receivable by the automatic user recognizer 14 and is transmitted.

For example, when the automatic user recognizer 14 supports a BLE protocol to perform short-range wireless communication with a mobile terminal, such as a smartphone, the mat identification unit 16 transmits the unique identification information carried on a Bluetooth signal.

The mat identification units 16 may be mounted on the array controllers of the unit mat sensors C1 to Cn, and the unique identification signal is received by the automatic user recognizer 14 to be described below, and then is provided to the walking analysis module 13.

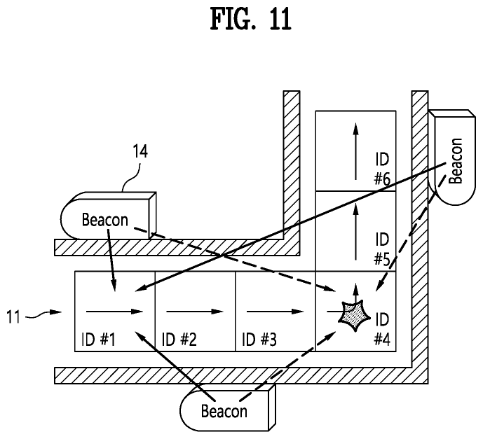

As shown in FIG. 11, in a case in which the mat identification unit 16 is further included, when the plurality of unit mat sensors C1 to Cn are assembled to form a walking path for measurement, the mats being used for walking may be identified by referring to the unique identification information. For example, the mat identification units 16 indicate that the unit mat sensors C1 to Cn used for walking correspond to ID #1 to ID #6.

That is, the history of unit mat sensors on which the subject is walking among the currently assembled unit mat sensors C1 to Cn is identified. Accordingly, the walking analysis module 13 may match identification information of the subject with identification information of the unit mat sensor so that the walking test progress position of the subject is read.

In addition, the automatic user recognizer 14 may analyze signals transmitted from the mat identification units 16 of each of the unit mat sensors C1 to Cn, to provide relative position information between the automatic user recognizer 14 and the unit mat sensors C1 to Cn.

The method of analyzing the transmitted signal to read the relative positions of the unit mat sensors C1 to Cn may include a method using an RSSI or a triangulation method.

Accordingly, the walking analysis module 13 may reflect the arrangement order of the unit mat sensors C1 to Cn read from the relative position information with the unit mat sensors C1 to Cn provided from the automatic user recognizer 14 in analyzing the walking state of the subject.

For example, when the unit mat sensors C1 to Cn are sequentially arranged from ID #1 to ID #6, it is read that the subject is sequentially walking along the corresponding unit mat sensors, based on which the foot pressure is analyzed to perform fall prediction and prevention analysis on the subject.

Next, the mat-side recognizer 17 is provided to recognize the subject, and uses short-range wireless communication, such as the BLE protocol, to recognize a mobile terminal possessed by the subject or a wearable device worn by the subject, to recognize an approach of the subject. The recognized information is collected and relayed through the automatic user recognizer 14.

The mat-side recognizers 17, as an embodiment, are installed on the unit mat sensors arranged at a position at which the subject enters for testing (measurement) and a position at which the subject leaves after completing the test among the unit mat sensors C1 to Cn.

In addition, when the plurality of unit mat sensors C1 to Cn are assembled and the walking distance becomes long or complicated, the mat-side recognizer 17 may be provided in the unit mat sensors C1 to Cn disposed in the middle between the beginning and the end of the movement path.

In particular, the mat-side recognizer 17 is installed in the unit mat sensors C1 to Cn disposed at a direction change point present between the entry point and the exit point of the subject, and with such a configuration, the walking test may be performed by reflecting changes in direction of the subject. In addition, the assembly pattern or arrangement direction of the unit mat sensors C1 to Cn may be analyzed.

Specifically, when a subject walking through the unit mat sensors C1 to Cn provided with the mat-side recognizer 17 is detected, the mat-side recognizer 17 transmits a mat passing signal to the walking analysis module 13 by a relay of the automatic user recognizer 14.

The walking analysis module 13 having received the mat passing signal may analyze a pressure sensor sensing signal at the time when the signal is input, and in particular, when the subject is passing through the unit mat sensors C1 to Cn disposed at the direction change point, this may be reflected in the analysis.

In FIG. 11, an example in which a unit mat sensor of ID #4 among the unit mat sensors C1 to Cn of ID #1 to ID #6 is disposed at a direction change point and arrangement of the unit mat sensors is curved upward based on the unit mat sensor of ID #4 is described.

Therefore, even when a walking state of the subject on the unit mat sensor of ID #4 abruptly changes, the walking analysis module 13 may recognize the change as a change in direction and reflect the change in direction in measuring the walking state used for fall prediction and prevention.

Hereinafter, a fall prevention management system according to the present invention will be described. The fall prevention management system of the present invention includes the hybrid walking analysis apparatus 10, the integrated database 20 and the app management module 30 as described above with reference to FIG. 1.

In this case, in order to predict and prevent a fall of the elderly person, the hybrid walking analysis apparatus 10 measures the foot pressure during walking using the mat-type walking analyzer 11 and acquires skeletal information using the depth camera 12 installed on the walking path.

Therefore, through skeletal recognition during walking of a subject, various walking patterns and walking postures during walking, including the skeleton, joints, left-right balance, leg angle, and foot height, may be analyzed.

Personal information and fall test history information of a subject are recorded in the integrated database 20, and the integrated database 20 provides a user/equipment authentication management function. Therefore, an integrated measurement information analysis data provision system is provided through management of user-specific fall test histories.

Furthermore, the integrated database 20 provides a customized fall prevention program suitable for a subject. The fall prevention program is provided by selecting the optimal prevention program in consideration of fall prediction and prevention results for each group through measurement of a walking state.

Prevention programs may be classified into various categories to prevent not only physical risks from circulatory, nervous system, musculoskeletal and visual factor, but also risks associated with aging, risks from medications ingested, risks from residential environment factors, and risks from lifestyle habits, such as drinking.

The app management module 30 provides an application for a terminal apparatus of a subject to access the integrated database and receive a test result. Therefore, in the present invention, the app management module 30 functions as an app download server.

Applications provided by the app management module 30 include various service environments, such as activation of an automatic user recognition function for a walking test, confirmation of a walking measurement result, and generation of an interface for requesting a fall prevention program.

Particular embodiments of the present invention have been described above. However, a person of ordinary skill in the art should appreciate that that there is no intention to limit the spirit and scope of the present invention to the particular embodiments disclosed and various modifications, equivalents, and other embodiments are possible without departing from the scope and sprit of the present invention.

Therefore, the embodiments described above are provided only to assist those of ordinary skill in the art in fully understanding the scope of the present invention, and should be construed as being illustrative rather than limiting the present invention, and the scope of the present invention is defined by the appended claims of the present invention.

### DESCRIPTION OF REFERENCE NUMERALS

10: hybrid walking analysis apparatus
11: walking analyzer (mat-type sensor)
12: 3D depth camera
13: walking analysis module
14: automatic user recognizer
15: 2D inspection camera
16: mat identification unit
17: mat-side recognizer
20: unified database
30: app management module

1. A hybrid walking analysis apparatus for fall prevention, the apparatus comprising:
a walking analyzer installed on a floor on which a subject of a fall prevention test walks is configured to walk and provided with comprising a plurality of pressure sensors for measuring a foot pressure during walking by the subject to provide foot pressure information;
a depth camera installed to photograph the walking subject and providing a three-dimensional (3D) depth image of the subject; and
a walking analysis module configured to aggregate the foot pressure information of the subject and skeletal information of the subject read from the 3D depth image and analyze a walking state of the subject.

2. The hybrid walking analysis apparatus of claim 1, further comprising an automatic user recognizer configured to determine a position of the subject to analyze each of a point in time when the subject enters the walking analyzer and a point in time when the subject leaves the walking analyzer.

3. The hybrid walking analysis apparatus of claim 2, wherein the walking analyzer comprises a plurality of unit mat sensors,
wherein each of plurality of the unit mat sensors (C1 to Cn) includes the plurality of pressure sensors arranged in a matrix pattern.

4. The hybrid walking analysis apparatus of claim 3, wherein the each of the unit mat sensors comprises:
a floor sheet;
a first sensor sheet stacked on an upper surface of the floor sheet and having a horizontal axis pressure sensor disposed in a horizontal direction;
a conductive material layer stacked on an upper surface of the first sensor sheet and formed of a conductive material;
a second sensor sheet stacked on an upper surface of the conductive material layer and having a vertical layer pressure sensor disposed in a vertical direction to intersect the horizontal axis pressure sensor; and
a finishing sheet stacked on an upper surface of the second sensor sheet.

5. The hybrid walking analysis apparatus of claim 1, wherein the depth camera is installed along the walking analyzer and is configured to continuously photograph the subject within designated sections.

6. The hybrid walking analysis apparatus of claim 5, further comprising a two-dimensional (2D) inspection camera configured to photograph the subject as a 2D red-green-blue (RGB) image and provide the 2D RGB image to the walking analysis module,
wherein the walking analysis module is configured to extract, among image frames in which the subject is photographed, an error frame that is different from an actual walking state using an analysis result of the 3D depth image provided by the depth camera and an analysis result of the 2D RGB image provided by the inspection camera and perform interpolation.

7. The hybrid walking analysis apparatus of claim 1, wherein the walking analysis module is configured to extract images of a skeleton and joints of the subject from an image frame through machine learning, wherein the walking state of the subject including a position value and an orientation value of the skeleton and the joints is analyzed.

8. The hybrid walking analysis apparatus of claim 7, wherein the walking analysis module comprises:
an object extraction unit configured to extract a walking aid used by the subject during walking from the 3D depth image;
a noise removing unit configured to exclude the extracted walking aid from the image frame to be analyzed;
a walking analysis unit configured to analyze the walking state of the subject using the image frame from which the walking aid is excluded; and
a fall prediction unit configured to compare the walking state of the subject with a fall risk factor to predict a risk of falling.

9. The hybrid walking analysis apparatus of claim 3, wherein the each of the plurality of unit mat sensors further comprises a mat identification unit that allows unique identification information thereof to be transmitted,
wherein the unique identification information of the mat identification unit is transmitted as a data packet conforming to a communication protocol that is receivable by the automatic user recognizer, and
wherein the walking analysis module is configured to match identification information of the subject with the unique identification information of the unit mat sensor to read a walking test progress position of the subject.

10. The hybrid walking analysis apparatus of claim 9, wherein the automatic user recognizer is configured to analyze signals transmitted from the each of the unit mat sensors to provide relative position information between the automatic user recognizer and the each of the unit mat sensors, and
wherein the walking analysis module is configured to reflect an arrangement order of the plurality of unit mat sensors read from the relative position information and analyze the walking state of the subject.

11. The hybrid walking analysis apparatus of claim 10, further comprising a mat-side recognizer installed among the plurality of unit mat sensors at a direction change point between an entry point at which the subject enters for testing and an exit point at which the subject leaves after completing the test,
wherein, when the subject walking through one of the plurality of unit mat sensors provided with the mat-side recognizer is detected, the mat-side recognizer is configured to transmit a mat passing signal to the walking analysis module by a relay of the automatic user recognizer, and
wherein the walking analysis module is configured to reflect information measured while the subject is walking through the direction change point in analyzing the walking state of the subject.

12. A fall prevention management system comprising:
the hybrid walking analysis apparatus of claim 1;
an integrated database configured to record personal information and fall test history information of the subject; and
an app management module configured to provide an application that allows a terminal device of the subject to access the integrated database and receive a test result.

13. The fall prevention management system of claim 12, wherein the integrated database is configured to provide the subject with a customized fall prevention program suitable for the subject.
